(19) United States
(12) Patent Application Publication
(10) Pub. No.: US 2009/0264375 A1
(43) Pub. Date: Oct. 22, 2009

(54) INTRAOCULAR IRRIGATING SOLUTIONS AND METHODS FOR TREATING CORNEAL EDEMA

(75) Inventors: Mandar V. SHAH, Rockaway, NJ (US); Joseph Paul BULLOCK, Fort Worth, TX (US); Alan L. WEINER, Arlington, TX (US); Harli DOLLINGER, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/339,812

(22) Filed: Dec. 19, 2008

Related U.S. Application Data
(60) Provisional application No. 61/016,166, filed on Dec. 21, 2007.
(60) Provisional application No. 61/016,197, filed on Dec. 21, 2007.

(51) Int. Cl. A61K 31/715; A61K 31/4172; A61K 31/66; A61K 31/7004

(52) U.S. Cl. 514/23; 514/400; 514/129; 514/54

(57) ABSTRACT

One aspect of the present invention relates to methods of treating corneal edema comprising contacting a cornea with an ophthalmic irrigating composition comprising histidine. In certain embodiments, the ophthalmic irrigating compositions contact the corneal endothelium. Another aspect of the present invention relates to ophthalmic irrigating compositions for treating corneal edema comprising histidine and optionally, calcium glycerophosphate and/or glutathione disulfide.

### CROSS-REFERENCE TO RELATED APPLICATION

This application claims priority under 35 U.S.C. §119 to U.S. Provisional Patent Application No. 61/016,166, filed Dec. 21, 2007, and to U.S. Provisional Patent Application No. 61/016,197, filed Dec. 21, 2007, the entire contents of which are incorporated herein by reference.

### TECHNICAL FIELD OF THE INVENTION

The present invention relates generally to surgical irrigating solutions and more specifically to intraocular irrigating solutions comprising histidine.

### BACKGROUND OF THE INVENTION

Ophthalmic surgical procedures, such as cataract surgery and vitrectomy surgery, often involve the very fragile tissues of the cornea and accordingly can damage the vision of a patient if the cornea is not properly cared for during the procedure. The cornea is a layered tissue structure comprising a thick layer of collagen stroma sandwiched between epithelial and endothelial layers. The interior endothelial layer is formed by a single-width layer of non-regenerating cells and is extremely delicate. The stromal tissue is maintained in a dehydrated state by fluid transport pumps in the endothelial layer and by the physical barriers to fluid influx formed by the epithelial and endothelial layers. Unfortunately, damage to either the epithelial or endothelial layer can allow fluid to leak into the stroma, and damage to the endothelial layer can interfere with the transport of fluid out of the stroma. Corneal edema is the undesirable swelling of the cornea caused by the buildup of fluid in the stromal and epithelial tissues. It can occur as a consequence of surgical insult, disease states, and physical and chemical trauma to the cornea.

Some previous treatments for corneal edema have incorporated the topical application of hypertonic solutions to the cornea to draw out stromal fluid. Alcohol-containing solutions have also been used with similar osmotic effects. See, e.g., U.S. Pat. No. 4,201,706. The use of irrigating solutions with therapeutic agents for ophthalmic surgery has been proposed. See, e.g., U.S. Pat. No. 5,523,316. The use of histidine-containing compositions for the prophylactic treatment of ocular inflammation is described in U.S. Pat. No. 5,811,446. However, ocular inflammation, while able to cause corneal tissue damage that may eventually lead to corneal edema, has a distinctly different pathology than corneal edema itself. Thus, there have been no known previous reports of histidine being used to treat corneal edema, nor has a histidine-containing irrigating solution for the treatment of corneal edema been proposed.

As noted above, corneal tissues are fragile and critical to vision. Accordingly, to incorporate factors necessary for sustained metabolism by corneal endothelial cells during ophthalmic surgery, glutathione-bicarbonate-Ringer's solution (GBR) was developed in which sodium bicarbonate, glutathione disulfide (GSSG), dextrose and optionally adenosine are added to Ringer's solution. Bicarbonate, an energy source such as dextrose, and GSSG have been shown to be important factors in maintaining the structural integrity of endothelial cells. GBR has been shown to be effective in maintaining corneal thickness and endothelial cell integrity during surgical procedures involving perfusion of the eye for up to three hours.

Unfortunately, previous efforts to prepare GSSG-containing irrigating solutions prepackaged as a single solution at neutral pH have not been successful. GSSG is unstable over extended periods of time at a pH of above about 5 or at the high temperatures present during terminal steam sterilization procedures. Accordingly, GBR and other ophthalmic irrigating solutions are typically packaged as two or more parts that are mixed together just prior to use. In a multi-part solution, the parts containing GSSG typically have a more acidic pH to enhance GSSG stability.

BSS PLUS® Sterile Intraocular Irrigating Solution (Alcon Laboratories, Inc.) is a widely used ophthalmic surgical irrigating solution that comprises GSSG. Like GBR, BSS PLUS® Sterile Intraocular Irrigating Solution is a two-part solution; the parts are mixed together to form a single solution just prior to surgery. This mixing step can be inconvenient for a busy operating room. Furthermore, manufacturing two separate solutions entails extra costs compared to a one-part formulation. Development of a one-part irrigating solution that comprises GSSG is therefore very desirable.

In addition, the two parts of BSS PLUS® Sterile Intraocular Irrigating Solution (and GBR) cannot simply be combined and terminally steam sterilized. Doing so can cause (i) precipitation of the dissolved salts; (ii) caramelization of dextrose; and/or (iii) degradation of GSSG in the solution. Also, the final reconstituted solution must have a pH close to 7, which further decreases the stability of any GSSG in the final solution.

There have been previous attempts to make a one-part irrigating solution comparable in performance to GBR and BSS PLUS® Sterile Intraocular Irrigating Solution. EP1067907 B1 (Armitage, et al.) teaches the use of zwitterionic organic buffers such as N-(2-hydroxyethyl)piperazine-N′-(2-ethanesulfonic acid), commonly referred to as HEPES, to prevent the precipitation between calcium and bicarbonate ions. The formulations disclosed by Armitage, et al. do not contain components such as dextrose and GSSG that are known to be unstable when autoclaved or incorporated in physiological pH solutions. In contrast, embodiments of the present invention do not require the use of zwitterionic buffers to prevent undesirable precipitation of bicarbonate salts. Also, while bicarbonate salts are indeed prone to form precipitates, phosphate salts are even more likely to exhibit this behavior. The teachings of Armitage, et al. do not appear to provide a solution to the problem of phosphate precipitate formation and do not teach the use of GSSG.

Improved compositions and methods are needed to better protect corneal tissues during ophthalmic procedures and reduce the risk of damaging the cornea. The compositions of the present invention provide improved corneal protection and the opportunity to improve ophthalmic surgical procedures and increase the likelihood of successful surgical outcomes by reducing the risk of, alleviating, or eliminating corneal edema.

### BRIEF SUMMARY OF THE INVENTION

The present invention relates to histidine-containing solutions for the treatment of corneal edema and methods for accomplishing such treatment. In certain embodiments, such treatment may be prophylactic.

Certain embodiments of the present invention comprise methods for treating corneal edema comprising contacting a cornea with an ophthalmic irrigating composition comprising histidine. Such embodiments may optionally comprise contacting the corneal endothelium with such compositions.

Preferred embodiments of the present invention comprise histidine- and calcium glycerophosphate-containing surgical irrigating solutions. As shown herein, histidine-containing compositions of the present invention effectively treat corneal edema. Calcium glycerophosphate has also been shown to effectively treat corneal edema as described in the co-pending U.S. application Ser. No. 12/339,536 (Attorney Docket No. 2760 US) entitled “STABILIZED GLYCEROPHOSPHATE-CONTAINING SURGICAL IRRIGATING SOLUTION,” filed on Dec. 19, 2008, the entire contents of which are hereby incorporated in the present specification by reference.

Certain formulations and methods of the present invention comprise glutathione disulfide (GSSG) and its use, the stability of which is improved by histidine. In one embodiment, the present invention relates to irrigating solutions comprising GSSG and a quantity of histidine sufficient to stabilize the GSSG. Another embodiment comprises bathing the intraocular tissues with an irrigating solution comprising GSSG and a quantity of histidine sufficient to stabilize the GSSG.

Certain solutions of the present invention optionally comprise carbohydrate energy sources such as sucrose or dextrose. Preferred solutions are terminally sterilized.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with specific embodiments described in the Examples portion of the specification. However, the Examples provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings and wherein:

FIG. 1 is a graph showing corneal perfusion data comparing a CaGP/histidine/sucrose formulation to a CaGP/histidine/GSSG/dextrose solution of the present invention; and

FIG. 2 is a graph showing corneal perfusion data comparing calcium glycerophosphate formulations having varying concentrations of histidine.

### DETAILED DESCRIPTION OF THE INVENTION

Histidine is a stereoisomeric essential amino acid. Embodiments of the present invention comprise histidine-containing irrigating solutions for the treatment of corneal edema. As used herein, the terms “treatment”, “treat”, and “treating” encompass the reduction or elimination of existing corneal edema, as well as prophylaxis to reduce the risk of causing or aggravating corneal edema. Embodiments of the present invention may contain histidine as either L-histidine or D-histidine, or may contain a racemic mixture of the two stereoisomeric histidine forms; L-histidine is preferred. The concentration of histidine in the solutions of the present invention should be between about 0.1 w/v % to about 1.0 w/v %, but is preferably about 0.7 w/v %.

The solutions of the present invention may further comprise a buffering system to maintain pH. A variety of buffering systems known to those of skill in the art may be used with embodiments of the invention. However, in some embodiments of the present invention, bicarbonate by itself or in combination with other compounds provides adequate buffering capacity to maintain pH. Citrate buffers may also be used with certain embodiments of the present invention.

The solutions of the present invention may further comprise carbohydrate energy sources, such as polysaccharides (e.g., sucrose) or monosaccharides (e.g., dextrose). However, when solutions comprising dextrose (such as BSS PLUS® Sterile Intraocular Irrigating Solution) are heat sterilized at a pH above 5, the dextrose tends to caramelize, forming an undesirable yellow color. Since a one-part irrigating solution can have only one pH which must be close to a physiologic pH, it is difficult to prevent dextrose from caramelizing during sterilization unless the solution is pH-adjusted after the sterilization. Sucrose is believed to be more resistant to carmelization during terminal steam sterilization than the dextrose found in BSS PLUS® Sterile Intraocular Irrigating Solution. Thus, sucrose is a preferred polysaccharide energy source for use with certain embodiments of the present invention.

Bicarbonate is a physiological buffer for the eye and bicarbonate salts are recognized as key components of ophthalmic irrigating solutions. Thus, embodiments of the present invention may include one or more bicarbonate salts at various concentrations including, without limitation, salts such as sodium or potassium bicarbonate. Preferred embodiments of the present invention comprise sodium bicarbonate. The concentration range of bicarbonate may be from about 0.1 w/v % to about 1.0 w/v % and the most preferred concentration is about 0.21 w/v %+10% (0.021 w/v %) excess.

It is desirable to include a calcium ion source in irrigating solutions for use in ophthalmic surgery, as increased calcium concentrations can prevent or reduce corneal edema during surgery. The solutions of the present invention may comprise calcium glycerophosphate (CaGP) as a calcium source. However, other calcium salts including, without limitation, calcium chloride, sulfate, acetate, citrate, lactate, and gluconate may be used in certain embodiments of the present invention at concentrations that may be determined by those of skill in the art. If CaGP is used, the CaGP concentration should be as high as possible without causing any precipitation in the irrigating solution. The preferred concentration of CaGP is from about 0.01 w/v % to about 0.5 w/v %, with a most preferred concentration of about 0.06 w/v %.

Glutathione disulfide (GSSG) is optionally present in the solutions of the present invention at concentrations ranging from 0.001 to 0.1 w/v %. A preferred range is 0.01 to 0.05 w/v %. GSSG is most preferred at a concentration of about 0.0184 w/v %+25% (0.0046 w/v %) excess.

The solutions of the present invention may also contain essential ions such as sodium, potassium, and chloride. Potassium and sodium may be provided in the form of various sodium and potassium salts known to those of skill in the art, such as sodium or potassium chlorides, sulfates, acetates, citrates, lactates, and gluconates. Similarly, chloride salts, such as sodium chloride and potassium chloride, may be used to provide chloride in solutions of the present invention. For the essential ions, the concentration of potassium should be about 0.01 w/v % to about 0.5 w/v %, with the most preferred concentration about 0.04 w/v %. The concentration of sodium should be about 0.1 w/v % to about 1.0 w/v %, with the most preferred concentration about 0.55 w/v %.

The most preferred surgical irrigating solutions of the present invention contain GSSG and histidine; CaGP as a source of calcium; bicarbonate as physiological buffer; essential ions such as sodium, potassium, and chloride; and optionally sucrose as an energy source.

As discussed above, a key advantage of certain formulations of the present invention is their ability to be terminally heat sterilized following addition and mixing of formulation ingredients without the undesirable reduction or elimination of GSSG in the solution. In preferred embodiments of the present invention, the formulation is prepared by mixing all ingredients and stirring until all components are in solution. The solution is then sterilized by dry or steam heat for a set time period (typically 30 minutes at 121° C.). However, the time and temperature of sterilization may vary and can be optimized by those of skill in the art.

The irrigating solutions of the present invention are suitable for use in a variety of ophthalmic and non-ophthalmic surgical procedures, but are particularly adapted and well-suited for use in conjunction with ophthalmic surgical procedures. The solutions are especially useful in conjunction with anterior chamber ophthalmic procedures that have the potential to expose the endothelial cells of the cornea. In other applications, the solutions may be used for foreign body removal and washing procedures. The solutions are suitable for posterior chamber procedures such as vitrectomy and for procedures involving the retina. The above list is not comprehensive and those of skill in the art will appreciate other applications for the disclosed embodiments of the present invention.

The Examples below were prepared according to embodiments of the present invention and are provided to further illustrate various features of the present invention.

### Example 1

| | Formulation Description | |
|---|---|---|
| | CaGP + histidine | |
| | (0.7%) + GSSG + | CaGP + histidine |
| | dextrose | (0.7%) + sucrose |
| Ingredients | (% w/v) | (% w/v) |
| Sodium Chloride | 0.59 | 0.59 |
| Potassium Chloride | 0.04 | 0.04 |
| CaGP, hydrate | 0.06 | 0.06 |
| Dextrose | 0.092 | — |
| Sucrose | — | 0.2 |
| L-Histidine | 0.7 | 0.7 |
| GSSG | 0.0184 + 25% excess | — |
| Sodium Bicarbonate | 0.21 + 10% excess | 0.21 + 10% excess |
| Water for Injection | q.s. to 100 | q.s. to 100 |
| F₀ for steam sterilization | 30 | 30 |
| Appearance after steam | Pale yellow | Colorless ^{d} |
| sterilization | | |
| Increase in corneal thick- | 10 μm (n = 3) | 5 μm (n = 4) |
| ness with test solutions ^{a} | | |
| Increase in corneal thick- | 33 μm (n = 2) ^{b} | 20 μm (n = 4) ^{c} |
| ness with control solutions | | |

^{a} After 3 hours of perfusion; n = number of rabbit corneas.
^{b} BSS PLUS ® Sterile Intraocular Irrigating Solution.
^{c} BSS ® Sterile Irrigating Solution.
^{d} After approximately one year, solution turned a pale greenish-yellow color.

### Example 2

| Ingredient | (% w/v) |
|---|---|
| GSSG | 0.0184 + 25% excess |
| L-Histidine | 0.4 |
| Sodium Chloride | 0.714 |
| Potassium Chloride | 0.038 |
| Calcium Chloride, dihydrate | 0.0154 |
| Magnesium Chloride, hexahydrate | 0.02 |
| Sodium Bicarbonate | 0.21 + 10% excess |
| Dextrose | 0.092 |
| Sodium Citrate, dihydrate | 0.2 |
| Hydrochloric Acid and/or | Adjust to pH 6.8 |
| Sodium Hydroxide | |
| Water for Injection | q.s. to 100 |
| F₀ for steam sterilization | 8 |
| Appearance after steam sterilization | No precipitate |

### Example 3

| | Formulation Description | | | |
|---|---|---|---|---|
| | Histidine | Histidine | Histidine | Histidine |
| | (0.0%) | (0.4%) | (0.7%) | (1.0%) |
| Ingredients | (% w/v) | (% w/v) | (% w/v) | (% w/v) |
| Sodium Chloride | 0.69 | 0.61 | 0.55 | 0.49 |
| Potassium Chloride | 0.04 | 0.04 | 0.04 | 0.04 |
| Sodium Citrate, | 0.2 | 0.2 | 0.2 | 0.2 |
| dihydrate | | | | |
| CaGP, hydrate | 0.06 | 0.06 | 0.06 | 0.06 |
| L-Histidine | — | 0.4 | 0.7 | 1.0 |
| Sodium Bicarbonate | 0.21 + 10% xs | 0.21 + 10% xs | 0.21 + 10% xs | 0.21 + 10% xs |
| Water for Injection | q.s. to 100 | q.s. to 100 | q.s. to 100 | q.s. to 100 |
| F₀ for steam | 30 | 30 | 30 | 30 |
| sterilization | | | | |
| Appearance after | Colorless | Colorless | Colorless | Colorless |
| steam sterilization | | | | |
| Increase in corneal | 28 μm (n = 2) | 18 μm (n = 4) | −16 μm (n = 2) | −24 μm (n = 2) |
| thickness with test | | | cornea shrinks | cornea shrinks |
| solutions ^{a} | | | | |
| Increase in corneal | 35 μm (n = 2) ^{b} | 103 μm (n = 4) ^{c} | 21 μm (n = 2) ^{b} | 27 μm (n = 2) ^{b} |
| thickness with | | | | |
| control solutions | | | | |

^{a} After 3 hours of perfusion; n = number of rabbit corneas.
^{b} BSS PLUS ® Sterile Intraocular Irrigating Solution.
^{c} BSS ® Sterile Irrigating Solution.

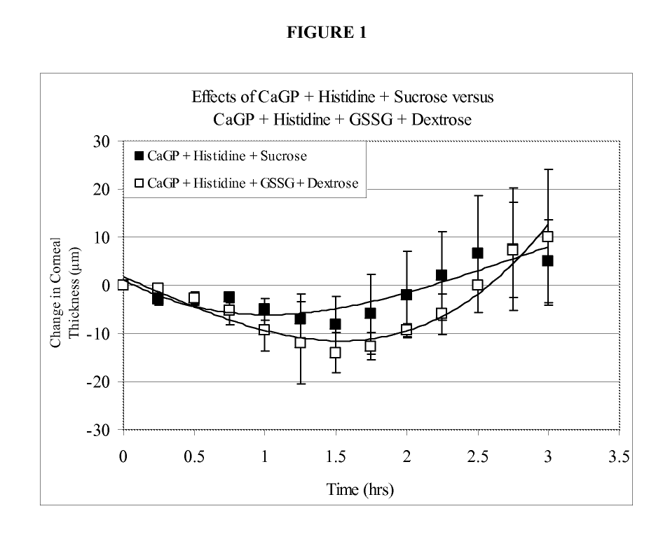

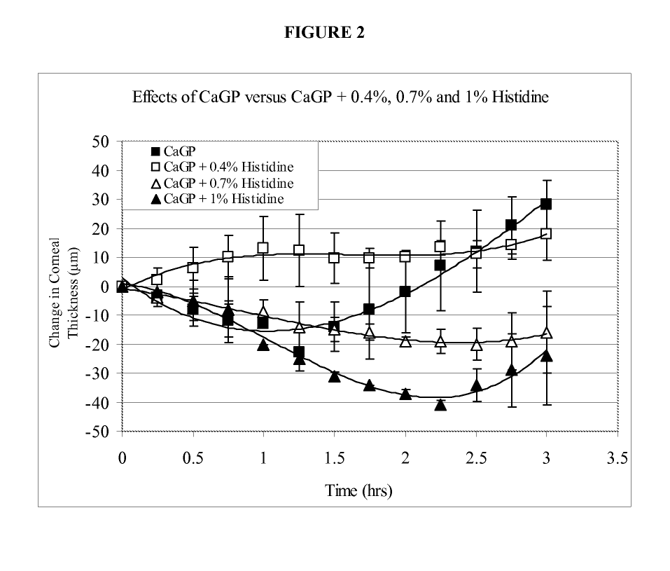

Corneal thickness changes in Examples 1 and 3 were determined using the rabbit corneal perfusion model, in which paired corneas of New Zealand White rabbits were isolated and mounted in an in vitro dual-chambered specular microscope designed for endothelial perfusion evaluation. Corneal thickness readings were taken at 15-minute intervals with the specular microscope for the entire length of perfusion, which lasted for 3 three hours. The corneal thickness data for the solutions of Examples 1 and 3 are presented graphically in FIGS. 1 and 2, respectively.

The composition of reconstituted BSS PLUS® Sterile Intraocular Irrigating Solution is shown below in Table 1. The reconstituted product was used as a control solution. A sample of the reconstituted product was then spiked with 0.4% histidine to form the experimental solution. The control solution and the experimental solution were then each divided into two replicates each. The pH of one control and one experimental replicate was then adjusted to pH 6.8 and the pH of the other replicate was adjusted to pH 7.4. Both sets of the solutions were then autoclaved at F₀=8. After autoclaving, GSSG contents in all the solutions were determined and the results are tabulated in Table 2 as percent GSSG remaining of the label claim in BSS PLUS® Sterile Intraocular Irrigating Solution.

TABLE 1

| Ingredient | (% w/v) |
|---|---|
| Sodium Chloride | 0.714 |
| Potassium Chloride | 0.038 |
| Calcium Chloride, dihydrate | 0.0154 |
| Magnesium Chloride, hexahydrate | 0.02 |
| Sodium Bicarbonate | 0.21 |
| GSSG | 0.0184 |
| Dextrose | 0.092 |
| Dibasic Sodium Phosphate | 0.042 |
| Hydrochloric Acid and/or Sodium Hydroxide | adjust to pH 7.4 |
| Water for Injection | q.s. to 100 |

TABLE 2

| | GSSG Concentration % of Label After Autoclaving | |
|---|---|---|
| | Reconstituted BSS PLUS ® | Reconstituted BSS PLUS ® |
| | Sterile Intraocular Irrigating | Sterile Intraocular Irrigating |
| pH | Solution (control) | Solution (with histidine) |
| 6.8 | 31 | 48 |
| 7.4 | 17 | 39 |

All of the above solutions were autoclaved in the same cycle. However, in this particular experiment, the sterilizer used was not capable of finely regulating F₀ and thus significant degradation of GSSG occurred. However, based on the above results, it is clear that GSSG is more stable at pH 6.8 than at pH 7.4 and that histidine stabilizes GSSG in solution.

For the next set of experiments, summarized in Table 3, the solutions were autoclaved in a sterilizer where F₀ can be more carefully controlled. The sterilizer, which had a water immersion cycle to reduce the temperature of the solutions quickly at the end of the sterilization cycle to prevent further exposure to heat, was set at F₀=6.5.

TABLE 3

| | GSSG Concentration % of Label After Autoclaving at F₀ = 6.5 | |
|---|---|---|
| | Reconstituted BSS PLUS ® | Reconstituted BSS PLUS ® |
| | Sterile Irrigating Solution | Sterile Irrigating Solution |
| pH | (control) | (with histidine) |
| 6.8 | 86, 85 | 99, 100 |
| 7.4 | 78, 78 | — ^{a} |

^{a} Assay was not performed.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

### REFERENCES

The following references, to the extent that they provide exemplary procedural or other details supplementary to those set forth herein, are specifically incorporated herein by reference.
Levenson J E, “Corneal Edema: Cause and Treatment”, Survey of Opthalmology, Vol. 20(3):190-204, November-December 975.
Liesegang T J, “The response of the corneal endothelium to intraocular surgery”, Refractive Corneal Surgery, Vol. 7(1):81-6, January-February 1991.
MacRae S, “Intraocular drugs used in cataract surgery and their effect on the corneal endothelium”, Refractive Corneal Surgery, Vol. 7(3):249-51, May-June 1991.
Hyndiuk, R A et al., “Overview of the corneal toxicity of surgical solutions and drugs; and clinical concepts in corneal edema”, Lens Eye Toxic Research, Vol. 9(3-4):331-50, 1992.

What is claimed is:

1. A method of treating corneal edema comprising:
contacting a cornea with an ophthalmic irrigating composition comprising an effective amount of histidine.

2. A method of claim 1 wherein said composition comprises histidine at a concentration of about 0.1 w/v % to about 1.0 w/v %.

3. A method of claim 1 wherein said composition comprises histidine at about 0.7 w/v %.

4. A method of claim 1 wherein said composition further comprises a energy source selected from the group consisting of:
polysaccharide, monosaccharide, and combinations thereof.

5. A method of claim 4 wherein said energy source is selected from the group consisting of:
sucrose, dextrose, and combinations thereof.

6. A method of claim 1 wherein said composition further comprises bicarbonate at a concentration of about 0.1 w/v % to about 1.0 w/v %.

7. A method of claim 6 wherein the concentration of said bicarbonate is about 0.21 w/v % plus an excess of 10% (0.021 w/v %).

8. A method of claim 1 wherein said composition further comprises calcium glycerophosphate at a concentration of about 0.01 w/v % to about 0.5 w/v %.

9. A method of claim 8 wherein the concentration of said calcium glycerophosphate is about 0.06 w/v %.

10. A method of claim 1 wherein said composition further comprises potassium at a concentration of about 0.01 w/v % to about 0.5 w/v % and sodium at a concentration of about 0.1 w/v % to about 1.0 w/v %.

11. A method of claim 10 wherein said composition comprises potassium at about 0.04 w/v % and sodium at a concentration of about 0.55 w/v %.

12. A method of claim 1 wherein said contacting comprises intracameral injection of said composition.

13. A method of claim 1 wherein said contacting comprises contacting the corneal endothelium.

14. A method of claim 1 wherein said composition further comprises glutathione disulfide at a concentration of about 0.01 w/v % to about 0.05 w/v %.

15. An ophthalmic irrigating composition for treating corneal edema comprising histidine and calcium glycerophosphate.

16. A composition of claim 15 further comprising glutathione disulfide at a concentration of about 0.01 w/v % to about 0.05 w/v %.

17. A composition of claim 15 comprising histidine at a concentration of about 0.1 w/v % to about 1.0 w/v %.

18. A composition of claim 15 wherein said composition comprises histidine at about 0.7 w/v %.

19. A composition of claim 15 comprising calcium glycerophosphate at a concentration of about 0.01 w/v % to about 0.5 w/v %.

20. A composition of claim 15 comprising calcium glycerophosphate at a concentration of about 0.06 w/v %.

21. A composition of claim 16 further comprising an energy source selected from the group consisting of:
polysaccharide, monosaccharide, and combinations thereof.
